(19) United States
(12) Patent Application Publication
(10) Pub. No.: US 2014/0073582 A1
(43) Pub. Date: Mar. 13, 2014

(54) INSECTICIDE CRY PROTEINS OF BACILLUS THURINGIENSIS WITH ANTI-CANCER ACTIVITY

(71) Applicants: Jorge Olmos Soto, Ensenada (MX); Efraín Arias Bañuelos, Ensenada (MX); Gretel Mendoza Almanza, Ensenada (MX)

(72) Inventors: Jorge Olmos Soto, Ensenada (MX); Efraín Arias Bañuelos, Ensenada (MX); Gretel Mendoza Almanza, Ensenada (MX)

(73) Assignee: Center for Scientific Research and Higher Education at Ensenda, Baja California, Carretera Ensenda-Tijuana (MX)

(21) Appl. No.: 13/994,687

(22) PCT Filed: Dec. 16, 2011
(86) PCT No.: PCT/IB11/55722

§ 371 Date: Aug. 21, 2013

(30) Foreign Application Priority Data
Dec. 17, 2010 (MX) MX/A/2011/000894

(51) Int. Cl. C07K 14/325

(52) U.S. Cl. CPC C07K 14/325
USPC 514/19.3; 530/350; 536/23.71; 435/252.31; 435/69.1

(57) ABSTRACT

The invention relates to nucleic acid molecules encoding insecticidal proteins of the Cry family, derived from Bacillus thuringiensis, exhibiting cytotoxic activity against cancer and/or tumor cells of humans and/or animals, but not against normal cells. The invention also provides proteins and compositions of proteins of the Cry family, derived from the bacteria Bacillus thuringiensis, that exhibit cytotoxic activity preferably against cancer cells of humans and/or animals, without affecting the normal cells. The Cry proteins of the invention do not exhibit any hemolytic activity, nor do they belong to the parasporin group. The invention also relates to methods for treating cancer and/or tumor cells of humans and/or animals, and for preventing metastasis, using insecticidal Cry proteins of Bacillus thuringiensis.

### TECHNICAL FIELD OF THE INVENTION

The present invention is related to the technical field of biomedicine and biotechnology, especially with the production and use of alternative molecules to treat cancer and more specifically, the use of insecticide Cry proteins derived from Bacillus thuringiensis (Bt) strains as molecules with cytotoxic activity against cancer cell lines (CCL), but without effects on non cancer cells (NCC). It is also related to methods to treat cancer cells utilizing these proteins and their derived pharmaceutical compositions.

### BACKGROUND OF THE INVENTION

Currently, it is known that cancer is one of the most devastating diseases in humans and it is originated by mistakes in gene regulation mechanisms that control cellular growth and proliferation. The mortality induced by cancer has increased over the past years therefore, a search for alternative treatments other than the ones used at present times, is necessary. Radiotherapy, chemotherapy and tumor removal, all have been shown to be effective on suppressing the disease only if they are applied on its first stage of development. In addition, their cost is high and adverse effects of therapies seriously jeopardize individual's health (INEGI, 2008). With the purpose of finding new cancer therapies that eliminates these inconveniences, a search for harmless biomolecules is being made. Some recently studied biomolecules have been demonstrated to have negative effects over cancer cells function, inhibiting cellular growth and proliferation, although a great majority of these land and marine molecules are difficult to produce, complicating clinical trials evaluation (Chu and Radhakrishnan, 2008).

Therefore, a constant need for new and specific biomolecules economically feasible to produce in great quantities that cure and/or eliminate cancer exists. Additionally, non-generation of adverse effects as severe as those induced by radiotherapy and chemotherapy are required.

Until the present no cure exists for cancer, that is why the search for new alternatives is intense. In this sense, the present invention gives a possible solution with successful results demonstrating to be more accurate than traditional therapies used.

Bacillus thuringiensis (Bt) is a gram positive bacterium with a polar flagellum, that measures 3 to 5 μm in length and 1 to 1.2 μm of width. It is a facultative anaerobic microorganism with catalase activity, that belongs to the Bacillaceae family and it is located within group 1 of Bacillus genus. Bt is characterized because in its sporulation process produces a parasporal inclusion constituted by one or more crystal shaped bodies of proteic nature, toxic for several invertebrates specially for insect larvae from Lepidoptera, Diptera and Coleoptera genera (Bravo and Güereca, 1998; Sauka and Benintende, 2008). These proteins are known as insecticidal crystal proteins (ICP) or δ-endotoxins. They are classified as Cry (45-160 kDa) and Cyt proteins (22-30 kDa), both proteins mainly differentiated by its insecticidal and hemolytic activity, respectively. Cry proteins are used in commercial insecticides worldwide (Bravo and Güereca, 1998; Rukmini et al., 1999; Sauka and Benintende, 2008; Sun et al., 2008; Yan Wu et al., 2008). While a great number of Cry proteins are toxic to insects, recent studies had demonstrated that other parasporal inclusions bodies from Bt lack insecticidal activity, even when they are submitted to in vitro evaluation simulating insect's conditions. Although these parasporal proteins are innocuous to insects, they present cytotoxic activity against human cancer cell lines (Ohba et al., 2009). These proteins have been named parasporines (PS), 13 have been reported and classified into 4 groups. PS neither posses insecticidal or hemolytic activity nor do they possess great sequence homology with Cry or Cyt proteins. Their cytotoxic activity, preferably against cancerous cells, makes PS possible candidates as anti-cancer agents for medical use, although at the present moment no clinical trials in human or animals have been made (Jung et al., 2007; Kitada et al., 2005; Mizuki et al., 2000, Nadarajah et al., 2006 and 2008; Ohba et al., 2009; Sauka and Benintende, 2008).

On the other hand, insecticide Cry proteins in its protoxin or toxin state does not have cytotoxic activity against NCC of human or animals, as described in documents U.S. Pat. No. 5,616,319, U.S. Pat. No. 5,985,267, US2005/0155112, AU652774 and as this invention. Additionally, prior to this invention no insecticide Cry protein has been reported to have cytotoxic activity against cancer cell lines.

In this sense, this invention provides insecticidal Cry proteins isolated from new Bt strains that produce cytotoxic activity against cancer cell lines but not against healthy cells. The Cry proteins tested are not parasporines (PS) neither do they have hemolytic activity as Cyt proteins do. Furthermore, Cry proteins from this invention show insecticidal activity due they belongs to the most important insecticide Cry protein groups. Anti-cancer activity is a property that up until now has not been described for an insecticide Cry protein of Bt.

### OBJECTIVES OF THE INVENTION

The present invention has as an objective to provide nucleic acid molecules (SEQ. ID. Nos. 1 to 10) from several new Bt strains, recollected from Baja California, Mexico region, which encodes proteins and fragments of insecticide Cry proteins, specially from groups 1, 2, 3 and 4, that presents a specific cytotoxic activity against cancer cells without affecting normal cells.

The present invention also has as an objective to provide new insecticide Cry proteins (SEQ. ID. Nos. 11 to 20) or its fragments that mainly belongs to 1, 2, 3, 4 Cry groups and presents specific cytotoxic activity against cancer cell without affecting normal cells.

Another objective of this invention is to contribute with compositions and/or formulations that contain Cry proteins of the present invention (SEQ. ID. Nos. 11 to 20) to treat cancer cells, where such compositions, besides containing Cry proteins from groups 1, 2, 3 and 4 preferentially, also contain combinations with other Cry proteins, as well as biological and/or chemical molecules that have anti cancerous activity.

Furthermore, the present invention also includes methods to cure and/or eliminate cancer in humans and/or animals, which include the application of an effective therapeutic quantity of the mentioned compositions, where the application can be made by any route of administration used in treatments against cancer.

Another goal of this invention is the use of Cry proteins and its mutants from groups 1, 2, 3, 4, and the proteins of the present invention (SEQ. ID. Nos. 11 to 20), as well as compositions that include pharmaceutical preparations, that will help to prevent, cure and/or eliminate cancer.

### BRIEF DESCRIPTION OF THE FIGURES

FIG. 1 Shows Cry protein electrophoresis in polyachrylamide gel. From left to right, (1) molecular weight marker, (2) protein profile of E strain obtained after crystal sonication, (3) same sample as in (2) plus solubilization process, (4) same sample as in (3) plus activation with trypsin, (5-9) protein profile of other trypsin activated Bt strains, (10) K trypsin activated strain.

FIG. 2 Shows effect of Cry proteins of the invention on MDA-MB-231 cancer cell line. (A), MDA-MB-231 cells without Cry proteins and (B) MDA-MB-231 cells with 1 μg/ml of Cry proteins produced by H strain. The cytotoxic effect can be identified by glowless circular morphology present in cells treated with Cry proteins.

FIG. 3 Shows effect of Cry proteins of the invention on MDA-MB-231 induced tumor. (a) nud mice with sixth day tumor, (b) same mice after 15 days application of Cry proteins produced by H strain, (c) same mice after 25 days application of Cry proteins produced by H strain.

### DETAILED DESCRIPTION OF THE INVENTION

The present invention provides Bt Cry proteins (SEQ. ID. Nos. 11 to 20) with anti-cancer activity, that can be used in therapy and/or treatment against cancer, as well as nucleic acid sequences that encode them (SEQ. ID. Nos. 1 to 10), making it possible to produce such proteins by known expression methods.

In one of its categories, the present invention also provides compositions that include Cry proteins that can be used to provide therapy and/or treatment to people affected by cancer. Furthermore, the present invention provides new applications using Cry proteins in therapy and/or treatment for cancer, especially those insecticide Cry proteins with cytotoxic activity against cancer cell but not against normal cells. Additionally, these Cry proteins must not be parasporines (PS) or hemolytic Cyt proteins.

In addition, Cry proteins herein described are encoded by nucleic acid sequences that can be potentially transcribed by methods known in the state of the art, either by sequences that show variations regarding the use of production system codons, but still capable of producing the protein of interest, allowing it to conserve its described properties and activity. Within these nucleic acid sequences are included those that show at least 80% homology with sequences of the invention (SEQ. ID. Nos. 1 to 10).

To determine effects of the invention, compositions here described comprise Cry proteins in therapeutically effective quantities, allowing to provide the mentioned anti-cancerous effects when they are administered to humans or animals, that need cancer treatment. In this sense, the administration regime will depend on several factors; including condition of the subject, the degree of cancerous advancement, type of treatment, administration technique and follow-up that the subject's medical specialist will provide. Additionally, compositions of the invention can be administered to subjects by several techniques in such a way that Cry proteins, including those described here (SEQ. ID. Nos. 11 to 20) can get to its target site. Furthermore, compositions of the invention described herein also include Cry proteins that are capable of providing anti-cancerous effects and at the same time, will not affect normal cells from people or animals.

The present invention provides new Cry proteins and/or its fragments that show anti-cancerous effects in such a way that when administered to animals affected with cancer and/or tumors, are capable of eliminating growth of such cancer and/or tumor without showing adverse effects to normal cells. Within these proteins or protein fragments are included those showing at least 80% homology with sequences of the invention (SEQ. ID. Nos. 11 to 20).

Therefore, administration of compositions of the present invention allows to provide effective therapies to humans or animals affected by cancer, for example, those affected with cancerous tumors; and at the same time they will not generate adverse effects with such treatment.

The present invention also describes methods for cancer treatment using compositions that include Cry proteins from Bt that show anti-cancerous activity, specially Cry proteins from groups 1 trough 4 and preferably Cry proteins from the present invention (SEQ. ID. Nos. 11 to 20). In this sense, previously mentioned Cry proteins can be used alone, as well as in mixtures of the same groups.

Cry proteins of the present invention can be comprised in an expression vector containing nucleic acid sequences that encodes at least one of them, allowing expression of such protein. In this sense, this vector may contain sequences that encode both chains of the Cry protein; therefore, such vectors containing Cry molecules of the invention can be contained in mammal cells, for example, human cells.

Cry proteins produced from this invention can be used to obtain pharmaceutical compositions, which can later on be administered to the subject of interest through known methods.

Compositions of the invention including previously described Cry molecules can be contained on several vehicles including liposomes, carriers, diluents and their salts. As well as acceptable pharmaceutical formulations needed for administration; such as tablets, sprays, solutions, micronized solids, injectable solutions, gels, creams, emulsions, lotions as well as other pharmaceutical presentations.

Compositions of the invention can be used in therapeutic methods or procedures to treat or prevent diseases, disorders or treat unfavorable health conditions related to cancer in humans or animals, including administration to such persons or animals with a therapeutically effective amount of such compositions under such conditions; allowing inhibition, elimination and/or progress of cancer. In this case, administration of compositions containing Cry molecules can be carry out by local or systemic ways (intravenous, intramuscular, subcutaneous or any other similar parentheral means), to tissues or cells that result relevant for the treatment. As well as the frequency (administration regime) and dosages also are important to achieved treatment. Otherwise, administration of compositions of the present invention can be combined with other treatments known in the state of the art to improve an individual's condition.

Consequently, methodology and characteristic details of the invention are described; the present description is accompanied by a series of tables and figures with the objective to make a better understanding of it, without limiting the reach of this invention.

### EXAMPLE 1

### Production of Cry Proteins in Bt Strains

Isolation and selection of Bacillus thuringiensis strain.

120 soil and water samples were collected from Baja California, Mexico region. Samples were treated by a spore selection method and were grown in Luria Bertani Medium (LB) for 24 hrs at 30° C. Colonies were isolated according to its Bt similar morphology described in LB medium. Selected colonies were grown in SP liquid medium and incubated at 30° C. for 96 hrs, at 200 rpm to induce Cry proteins production. Bacillus crystals producer strains presenting similar morphology to Bt were selected for molecular characterization.

Molecular Characterization of Bt Strains and Cry Genes.

DNA Purification

DNA isolation from identified and selected Bacillus strains was done using alkaline lysis and phenol-chloroform method (Sambrook et al., 1989). DNA integrity was verified in a 0.8% agarose gel exposed to ultraviolet light. 16S rDNA genes were amplified using universal oligonucleotides (Arellano and Olmos, 2002). Cry genes were also amplified by PCR technique, using specific oligos for each group (Table 1). PCR reactions were done using the following conditions: 2 min cycle at 95° C., 30 cycles of 1 min at 95° C., 1 min at indicated melting temperature for each oligonucleotide and 1 min at 72° C. One more cycle at 72° C. for 5 min. PCR products were submitted to electrophoresis using a molecular weight marker, positive samples were sequenced (SEQ. ID. Nos. 1 to 10) and subsequently analyzed by BLAST program to corroborate identity of Bt strains and to identify cry genes present in selected strains (Table 2).

TABLE 1
Specific oligonucleotides for cry genes amplification

| | | | | Size | |
|---|---|---|---|---|---|
| Name | Sequence¹ | Gene | T (° C.)² | (pb) | Reference³ |
| cry1A | 5′ATTCGCTAGGAACCAAGC (f) | cry1A | 55 | 398 | Thammasittirong |
| | 5′AATCCGGTCCCCATACAC (r) | | | | and Attathom |
| | | | | | 2008 |
| cry1B | 5′CTTCATCACGATGGAGTAA (f) | cry1B | 50 | 369 | EF102874.1 |
| | 5′CATAATTTGGTCGTTCTGTT (r) | | | | |
| cry1C | 5′CAAAGATCTGGAACACCTT (f) | cry1C | 50 | 131 | AY955268.1 |
| | 5′CAAACTCTAAATCCTTTCAC (r) | | | | |
| cry1D | 5′AAGGGAAGGAAATACAGAGC (f) | cry1D | 54 | 641 | Thammasittirong |
| | 5′CGAACGAACGAGATGTTAG (r) | | | | and Attathom |
| | | | | | 2008 |
| cry1E | 5′GAACCAAGACGAACTATTG (f) | crylE | 50 | 144 | M73252.1 |
| | 5′TGAATGAACCCTACTCCC (r) | | | | |
| cry1F | 5′GCAGGAAGTGATTCATGG (f) | cry1F | 50 | 432 | EU679501.1 |
| | 5′CAATGTGAATGTACTTTGCG (r) | | | | |
| cry2Aa | 5′CAAGCGAATATAAGGGAGT (f) | cry2Aa | 50 | 460 | AF273218.1 |
| | 5′TAGCGCCAGAAGATACCA (r) | | | | |
| cry2Ab | 5′CACCTGGTGGAGCACGAG (f) | cry2Ab | 50 | 771 | AF336115.1 |
| | 5′GTCTACGATGAATGTCCC (r) | | | | |
| cry3 | 5′TTAACCGTTTTCGCAGAGA (f) | cry3 | 50 | 713 | Ceron et al., |
| | 5′TCCGCACTTCTATGTGTCCAAG (r) | | | | 1995 |
| cry4 | 5′TCAAAGATCATTTCAAAATTACATG (f) | cry4 | 50 | 459 | Ibarra et al., |
| | 5′CGGCTTGATCTATGTCATAATCTGT (r) | | | | 2003 |

¹(f) forward; (r) reverse;
²(T) Alignment temperature

Table 2 shown Bt strains that amplified cry genes from groups; 1, 2, 3 and 4, considered the most important and abundant insecticidal groups, however, use of other Cry groups to treat cancer it is not discarded. It is important to mention that according to results obtained from 16S rDNA gene sequences, all selected strains were Bt species. Additionally, parasporines (PS) specific oligonucleotides were used to demonstrate that none of selected strains contained genes to produce this kind of proteins (Ohba et al., 2003). In this sense, obtained results assure that cytotoxic activity against cancer cells was exclusively generated by insecticide Cry proteins.

TABLE 2
PCR cry genes amplification from Bt selected strains

| Isolated Bt strains | cry genes amplified |
|---|---|
| A | cry1A |
| B | cry2Aa, cry2Ab |
| C | cry1A, cry1E, cry1F |
| D | cry1B, cry1C, cry1D |
| E | cry1A, cry2Aa, cry2Ab |
| F | cry1A, cry2Aa, cry2Ab |
| G | cry1A, cry1D, cry2Aa |
| H | cry1A, cry1D, cry2Aa, cry2Ab |
| I | cry3 |
| J | cry1A, cry3 |
| K | cry4 |
| L | cry1A, cry4 |

Strains that amplified cyt genes were excluded from the study because they contained hemolytic activity against human and animal erythrocytes, which was not desired for this study.

The sequence results from PCR products with specific oligonucleotides, proved identify of the amplified genes (SEQ. ID. Nos. 1 to 10).

Cry Proteins Production and Electrophoresis Evaluation

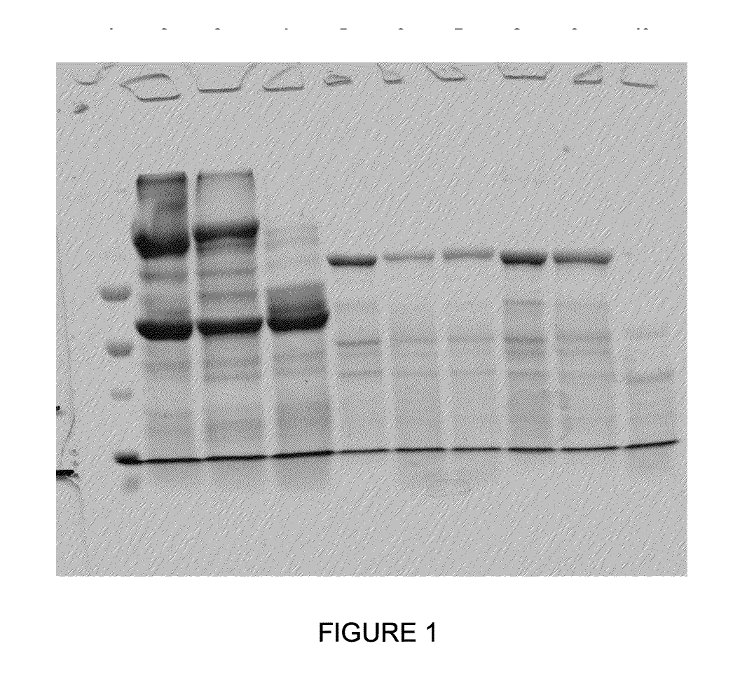

Once genes were identified, we proceeded to confirm production of insecticidal Cry proteins in selected Bt strains (Table 2). Strains were grown in SP liquid medium and obtained crystals were sonicated, solubilized and activated by enzymatic proteolysis. Treated protein samples were submitted to polyacrylamide gel electrophoresis. Insecticidal Cry proteins are characterized by containing 45 to 240 kDa bands, for groups 1, 2, 3 and 4. FIG. 1 shows protein profiles of some of the isolated strains; lanes 2, 3 and 4 represents sonicated, solubilized and trypsin activated crystals from E strain. In trypsin activated sample Cry1 and Cry2 proteins of 60 and 65 kDa respectively, can be observed.

Cry Proteins Trypsin Activation

Crystals produced from selected Bt strains were submitted to several treatments to activate protoxins contained in parasporal Bt inclusions. The solubilization was carried out once Bt strains were harvested from culture medium and washed. Pellets were resuspended in TTN buffer, incubated at 37° C. for 30 min and 6 min of sonication process. Sonicated samples were solubilized through an alkaline pH of 9 to 11, to obtain the protoxins. Protoxins were activated using trypsin at concentrations of 5 to 50 μg/ml, as well as different incubation time periods.

Activated toxins were filtered using a 0.2 μm pore membrane, with the purpose of eliminating all possible contamination from remaining bacteria or spores. The activated toxins were preserved at −20° C. for its future HPLC purification and utilization.

### EXAMPLE 2

### Cry Proteins Insecticidal Activity

Crystals produced in accordance to example 1 were washed and diluted to obtain 2 μg/cm² of Cry proteins concentration, that were used to test insecticidal activity in Manduca sexta. Toxicity evaluation was made in 24 well plates containing one larva per each well. Mentioned proteins concentration was added to food pellets that were incubated for 7 days. Table 3 shows insecticidal activity after 7 days incubation using selected Bt strains. Results shows that evaluated Cry proteins of the invention, either in groups or alone, presented an important insecticidal activity.

TABLE 3
Mortality percentage of Manduca sexta
larvae using Cry proteins of the invention

| Bt strain | Cry proteins produced | Mortality % |
|---|---|---|
| A | Cry1A | 100 |
| B | Cry2Aa, Cry2Ab | 50 |
| C | Cry1A, Cry1E, Cry1F | 100 |
| D | Cry1B, Cry1C, Cry1D | 75 |
| E | Cry1A, Cry2Aa, Cry2Ab | 100 |
| F | Cry1A, Cry2Aa, Cry2Ab | 100 |
| G | Cry1A, Cry1D, Cry2Aa, | 100 |
| H | Cry1A, Cry1D, Cry2Aa, | 100 |
| | Cry2Ab | |
| I | Cry3 | 50 |
| J | Cry1A, Cry3 | 100 |
| K | Cry4 | 50 |
| L | Cry1A, Cry4 | 100 |

### EXAMPLE 3

### Cry Proteins Cytotoxic Effect on Cancer Cell Lines

In Vitro Cytotoxicity Assay Using Cancer Cell Lines and Cry Proteins of the Invention

Cell Growth

Human keratinocytes cell line (HaCat) was used as a non-cancerous control and was cultivated in RPMI medium. Cervical cancer cell line (HeLa) and breast cancer cell line (MDA-MB-231), were cultivated in the same medium as HaCat. The medium was supplemented with 10% of bovine fetal serum and 1% of antibiotic and antifungal solution. Cultures were incubated at 37° C. with 5% of CO₂ and humidified atmosphere. Cells were maintained in growth by subculturing twice a week. Before doing assays with cancer cell lines, toxins of selected Bt strains were submitted to hemolytic activity tests using human erythrocytes, to discard hemolytic effects of Cyt proteins.

Cry Proteins Cytotoxic Cancer Cell Lines Effect

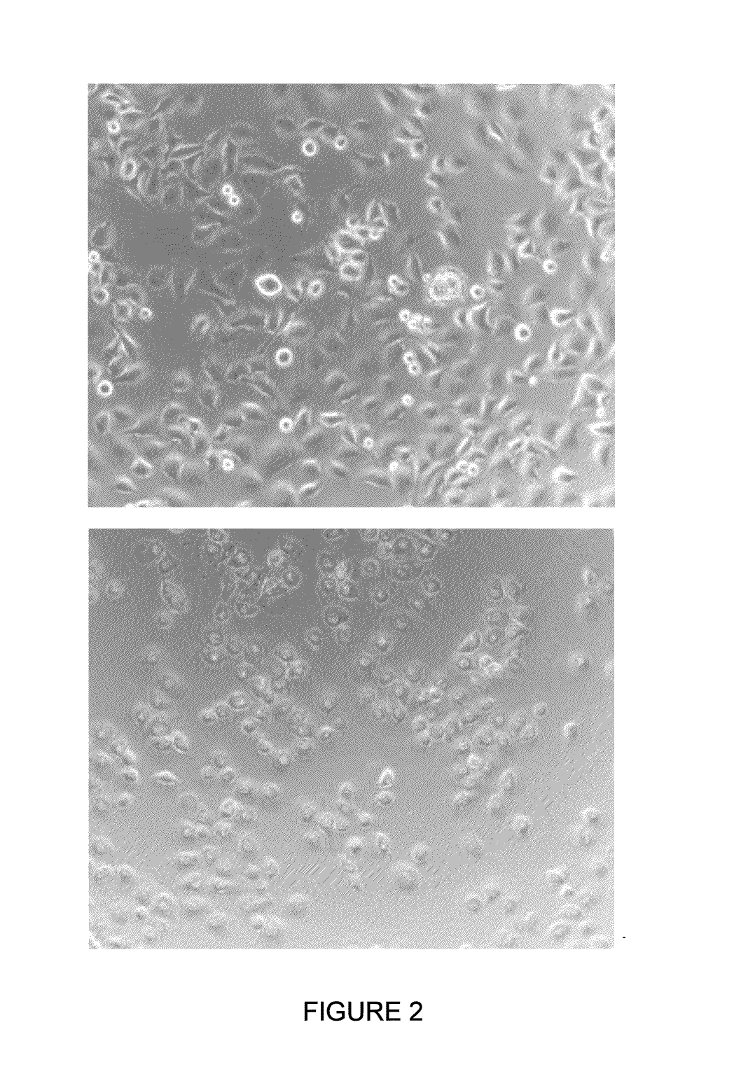

Micro culture plates of 96 wells with 100μl of supplemented medium containing 1×10⁴ cells per well were used. Plates were incubated 4 hrs at 37° C. and 5% of CO₂. After cell adhesion supplemented medium was discarded and the same amount of non-supplemented medium was added. After 24 hrs of incubation under the mentioned conditions, activated toxins were added in concentrations of 1.0, 0.5 and 0.25 μg/ml, each concentration was analyzed by triplicate. Four more hours of incubation were carried out and we proceeded to measure cellular viability by microscopy (FIG. 2) and MMT methodology. Table 4 shows cytotoxic activity of Cry proteins on cancer cell lines tested. Cry proteins that presented greater catatonic activity in HeLa and MDA and by consequence a greater percentage of mortality in these cancer cell lines, were those produced by E and H strains. However, most of the selected Bt strains of this invention, also presented an important catatonic activity against analyzed cancer cell lines (Table 4). In the particular case of HaCat which were used as a non-cancerous control cells, it was observed that none of the used insecticidal Cry proteins presented significant cytotoxic activity against them. These results confirm that activity of insecticidal Cry proteins of the invention was almost exclusive to cancer cell lines. Protoxins were used as another control in the assays with cancer cell lines, giving no cytotoxic activity as a result, demonstrating that protoxins must be trypsin activated to show their cytotoxic activity against cancer cell lines.

In U.S. Pat. No. 5,824,636 patent a Cyt protein was used at 100 μg/ml concentration to obtain cytotoxic activity against cancer cell lines. However, Cyt proteins are hemolytic and their use is not recommended in any human condition. Otherwise, US2003/0210317 and U.S. Pat. No. 7,329,733 patents describe the use of perspiring isolated from Bt strains without insecticidal activity, that presented good cytotoxic activity results at 0.5 μg/ml concentrations, specially for leukemia cells. In this sense, the present invention represents a new alternative to treat cancer cells, since at concentrations of 0.25 μg/ml, good results were obtained using insecticide Cry proteins that never have been used or reported previously for cancer cell lines treatment. It is important to point out that insecticidal Cry proteins of the present invention did not have any catatonic activity against utilized HaCat non-cancerous control cells.

TABLE 4
DL₅₀ of insecticidal Cry proteins

| | | DL₅₀ (pg/ml) | | |
|---|---|---|---|---|
| Strain | Cry proteins | HeLa | MDA | HaCat |
| A | Cry1A | 0.5 | 0.5 | <10 ND |
| B | Cry2Aa, Cry2Ab | 1.0 | 1.0 | <10 ND |
| C | Cry1A, Cry1E, | 0.5 | 1.0 | <10 ND |
| | Cry1F | | | |
| D | Cry1B, Cry1C, | 0.5 | 0.5 | <10 ND |
| | Cry1D | | | |
| E | Cry1A, Cry2Aa, | 0.25 | 0.25 | <10 ND |
| | Cry2Ab | | | |
| F | Cry1A, Cry2Aa, | 0.25 | 0.5 | <10 ND |
| | Cry2Ab | | | |
| G | Cry1A, Cry1D, | 0.5 | 1.0 | <10 ND |
| | Cry2Aa, | | | |
| H | Cry1A, Cry1D, | 0.25 | 0.25 | <10 ND |
| | Cry2Aa, Cry2Ab | | | |
| I | Cry3 | 1.00 | 0.5 | <10 ND |
| J | Cry1A, Cry3 | 0.5 | 0.25 | <10 ND |
| K | Cry4 | 1.00 | 1.00 | <10 ND |
| L | Cry1A, Cry4 | 0.5 | 0.5 | <10 ND |

ND. Not detected.

### EXAMPLE 4

### Cytotoxic Activity of Insecticidal Cry Proteins of the Invention in Nud Mice with Induced Tumors

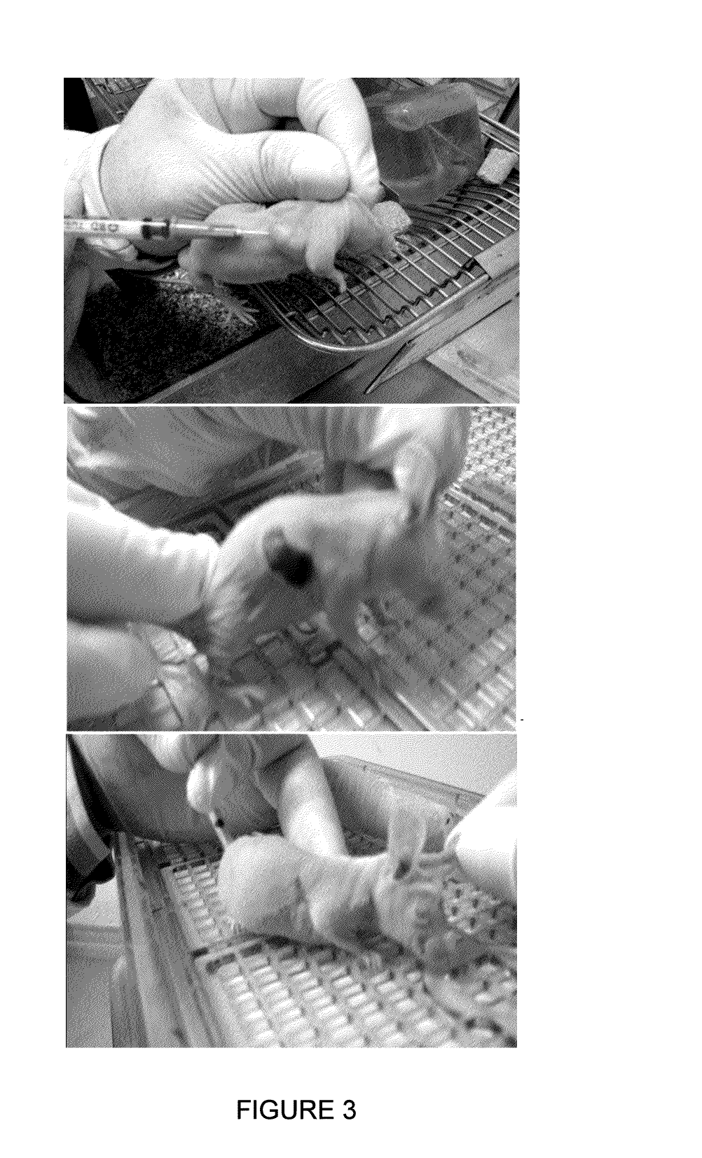

Cry proteins purified from E and H Bt strains were utilized to treat nud mice, which have the characteristic of being immunologically deficient. Tumors in mice were induced using HeLa and MDA cells that after 6 days of growth, presented an average size of 2 cm in diameter and 1 cm in height. At this time mice were inoculated directly in tumors with toxins from the strains mentioned above and their progress was followed each 5 days. After 25 days of Cry proteins inoculation successful results were obtained on tumor elimination and mice presented 100% recovery. No evidence were recorded of physical tumor and all vital signs and hematological parameters were normal (FIG. 3). These in-vivo results also demonstrate that utilized insecticidal Cry proteins of the invention did not affected normal or healthy cells, only cancer cells. The mice were evaluated for an additional 25 days to determine if there was a tumor regression, which did not occur with any of the evaluated samples. The mice experiment demonstrated that in fact insecticidal Cry proteins of the invention can be used to treat cancer in animals and humans. The cytotoxic activity obtained and showed in Table 4 as well as the results on nud mice are by far valuable and promising, due to the reason that for first time it has been demonstrated that Cry proteins cataloged as insecticides, have great potential to be used as anti-cancerous agents.

### BIBLIOGRAPHY

1. Aggarwall, B. and Rodríguez-Padilla, Cristina. 1998. Antiproliferative protein from Bacillus thuringiensis var. thuringiensis. U.S. Pat. No. 5,824,636.
2. Arellano-Carbajal, F. and Olmos, J. 2002. Thermostable α-1-4-and α-1-6-glucosidase enzymes from Bacillus sp isolated from a marine environment. World Journal of Microbiology and Biotechnology 18:791-795.
3. Bravo, A. and Güereca, L. 1998. The oligomeric state of Bacillus thuringiensis Cry toxins in solution. Biochimica and Biophysica 1429: 342-350.
4. Coté, J., Jung, Y., Mizuki, E. and Akao, T. 2008. Bacillus thuringiensis strain crystal gene and Crystal protein and uses thereof. U.S. Pat. No. 7,329,733 B2.
5. Donovan, W., Tan, Y., Jany, C. and González, José. 1997. Bacillus Thuringiensis CryEt5 Gene and related plasmids, bacteria and Insecticides. U.S. Pat. No. 5,616,319.
6. Hickle, L., Payne, J., Bradfisch, G. and Sick, August. 1994. The use of Bacillus thuringiensis microbes for controlling the lesser mealworm alphitobius diaperinus. AU652774(B2).
7. Jung, Y., Mizuki, E., Asao, T. and Coté J. 2007. Isolation and characterization of a novel Bacillus thuringiensis strain expressing a novel cristal protein with cytocidal activity against human cancer cells. Journal of Applied Microbiology 103, p. 65-79.
8. Kitada, S., Abe, Y., Ito, A., Kuge, O., Akao, T., Mizuki, E. and Ohba, M. (2005) Molecular Identification and Cytocidal Action of Parasporin, a Protein Group of Novel Crystal Toxins Targeting Human Cancer Cells. 6^{th} Pacific Rim Conference on the Biotechnology of Bacillus thuringiensis and its Environmental Impact. Victoria, B C.
9. Mizuki, E., Shin Park, Y., Saitoh, H., Yamashita, S., Akao, T., Higuchi, K. and Ohba, M. (2000) Parasporin, a Human Leukemic Cell-Recognizing Parasporal Protein of Bacillus Thuringiensis. Clinical and Diagnostic Laboratory Immunology, 625-634.
10. Nadarajah, V. D., Ting, D., Mohamed, S M., Kanakeswary, K and Lee, H L. (2008). Selective Cytotoxic Activity against Leukemic Cell Lines From Mosquitocidal Bacillus Thuringiensis Parasporal Inclusions. Human Biology Section. Faculty of Medicine, International Medical University.
11. Nadarajah, V. D., Chai, S. H., Mohamed, S M., Chan, K. K. and Kanakeswary, K. (2006). Malaysian Mosquitocidal Soil Bacterium (Bacillus thuringiensis) Strains with Selective Hemolytic and Lectin Activity Against Human and Rat Erythrocytes. Human Biology Section. Faculty of Medicine, International Medical University 37: no. 1.
12. Ohba, M., Mizuki, E. and Uemori, A. (2009). Parasporin, a new anticancer Protein Group from Bacillus thuringiensis. Anticancer Research 29: 427-434.
13. Ohba, M., Mizuki, E. and Uemori, A., Akao, T., Saito, H., Katayama, H., Yamashita, S. and Lee, D. Protein Having Cell recognizing activity and/or cytocidal activity. US2003/0216317 A1.
14. Payne, J., Sick, A., Narva, K., Schnepf, H. and Shwab, G. 1999. Protein toxins active against Lepidopteran pests. U.S. Pat. No. 5,985,267.
15. Rukmini, V., Reddy, C. and Venkateswerlu, G. 1999. Bacillus thuringiensis cristal-endotoxin: Role of proteases in the conversion of protoxin to toxin. Biochimie 82: 109-116.
16. Sambrook, K. T., Frisch, E. F. and Maniatis, T. (1989) Molecular cloning. A laboratory Manual. 2nd edn. Cold Spring Harbor Laboratory Press, Cold Spring Harbor, N.Y.
17. Sauka, D. and Benintende, G. (2008). Bacillus thuringiensis: generalidades. Un acercamiento a su empleo en el biocontrol de insectos lepidóptéros que son plagas agrícolas. Revista Argentina de Microbiología 40: 124-140.
18. Sun, Y., Fu, Z., Ding, X. and Xia, L. (2008) Evaluating the Insecticidal Genes and their Expressed Products in Bacillus Thuringiensis Strains by Combining PCR with Mass Spectrometry. Applied and Environmental Microbiology. P. 6811-6813.
19. Chu, W. L. and Radhakrishnan, A. (2008). Research on bioactive molecules: Achievements and the Way Forward. JSME Suppl 1, S21-S24.
20. Wu, Y., Gao, M., Dai, S., Yi, D. and Fan, H. (2008). Investigation of the cyt gene in Bacillus thuringiensis and the biological activities of Bt isolates from the soil of China. Biological Control 47; 335-339.

1. An isolated Cry protein of Bacillus thuringiensis with anti-cancer activity that exhibits specific cytotoxic activity against cancer cells and that is not cytotoxic to normal cells.

2. The Cry protein of claim 1 that is selected from among Cry1, Cry2, Cry3, and Cry4 protein groups and that comprises at least one sequence of amino acids selected from among SEQ. ID. No. 11 to SEQ. ID. No. 20, or that exhibits at least 80% homology to an amino acid sequence selected from among SEQ. ID. No. 11 to SEQ. ID. No. 20, and fragments or peptides derived from the proteins and their mixtures.

3. The Cry protein of claim 2, that comprises at least one sequence of amino acids selected from among SEQ. ID. No. 11 to SEQ. ID. No. 20.

4. The Cry protein of claim 1 that does not exhibit hemolytic activity.

5. A nucleic acid molecule that encodes the Cry protein of claim 1.

6. A nucleic acid molecule that encodes the Cry protein of claim 2.

7. A nucleic acid molecule that encodes the Cry protein of claim 3.

8. The nucleic acid molecule of claim 7 that comprises a sequence of nucleotides as set forth in any one of SEQ. ID. No. 1 to SEQ. ID. No. 10.

9. A molecular expression vehicle comprising a nucleic acid molecule of claim 5.

10. A host cell comprising the molecular expression vehicle of claim 9.

11. A pharmaceutical composition with anti-cancer activity, comprising a therapeutically effective amount of the Cry protein of claim 1; and
a pharmaceutically accepted vehicle.

12. A pharmaceutical composition with anti-cancer activity, comprising a therapeutically effective amount of the Cry protein of claim 3; and
a pharmaceutically accepted vehicle.

13. A method for treating cancer in animals and humans, comprising administrating a therapeutically effective amount of the composition of claims 11 to an animal or human in need thereof.

14. A method for treating cancer in animals and humans, comprising administrating a therapeutically effective amount of the composition of claim 12 to an animal or human in need thereof.

15. (canceled)

16. (canceled)

17. A method of preparing a Cry protein, comprising:
a) growing a host cell of claim 10 in a medium under conditions that allow the production of Cry proteins in a growth medium; and
b) purifying the Cry proteins from the growth medium.

18. A method of preparing a pharmaceutical composition, comprising:
mixing a therapeutically effective amount of a Cry protein of claim 1 with an acceptable pharmaceutical vehicle.

19. A pharmaceutical composition with anti-cancer activity, comprising:
a therapeutically effective amount of the Cry protein of claim 2; and
a pharmaceutically accepted vehicle.

20. A pharmaceutical composition with anti-cancer activity, comprising:
a therapeutically effective amount of the Cry protein of claim 4; and
a pharmaceutically accepted vehicle.

21. A molecular expression vehicle, comprising a nucleic acid molecule of claim 6.

22. A molecular expression vehicle, comprising a nucleic acid molecule of claim 7.


SEQUENCE LISTING (40 sequences)

SEQ ID NO: 1 - DNA, 398 bp, Bacillus thuringiensis
  misc_feature: Sequence cry1A of Bt from Baja California, Mexico
attcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctcttttggcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtgcgctggtacaatacgggattagagcgtgtatggggaccggatt

SEQ ID NO: 2 - DNA, 369 bp, Bacillus thuringiensis
  misc_feature: Sequence cry1B of Bt from Baja California, Mexico
cttcatcacgatggagtaatactaggcatatgacttattggcgggggcacacgattcaatctcggccaataggaggcggattaaatacctcaacgcatggggctaccaatacttctattaatcctgtaacattacggttcgcatctcgagacgtttataggactgaatcatatgcaggagtgcttctatggggaatttaccttgaacctattcatggtgtccctactgttaggtttaattttacgaaccctcagaatatttctgatagaggtaccgctaactatagtcaaccttatgagtcacctgggcttcaattaaaagattcagaaactgaattaccaccagaaacaacagaacgaccaaattatg

SEQ ID NO: 3 - DNA, 131 bp, Bacillus thuringiensis
  misc_feature: Sequence cry1C of Bt from Baja California, Mexico
caaagatctggaacaccttttttaacaactggtgtagtattttcttggacgcatcgtagtgcaactcttacaaatacaattgatccagagagaattaatcaaatacctttagtgaaaggatttagagtttg

SEQ ID NO: 4 - DNA, 641 bp, Bacillus thuringiensis
  misc_feature: Sequence cry1D of Bt from Baja California, Mexico
aagggaaggaaatacagagcgccccgtaactattaccgcatcacctagcgtaccaatatttagaacactttcatatattacaggccttgacaattcaaatcctgtagctggaatcgagggagtggaattccaaaatactataagtagaagtatctatcgtaaaagcggtccaatagattcttttagtgaattaccacctcaagatgccagcgtatctcctgcaattgggtatagtcaccgtttatgccatgcaacatttttagaacggattagtggaccaagaatagcaggcaccgtattttcttggacacaccgtagtgccagccctactaatgaagtaagtccatctagaattacacaaattccatgggtaaaggcgcatactcttgcatctggtgcctccgtcattaaaggtcctggatttacaggtggagatattctgactaggaatagtatgggcgagctggggaccttacgagtaaccttcacaggaagattaccacaaagttattatatacgtttccgttatgcttcggtagcaaataggagtggtacatttagatattcacagccaccttcgtatggaatttcatttccaaaaactatggacgcaggtgaaccactaacatctcgttcgttcg

SEQ ID NO: 5 - DNA, 143 bp, Bacillus thuringiensis
  misc_feature: Sequence cry1E of Bt from Baja California, Mexico
gaaccaagacgaactattgctcctagtacttttccaggtcttaacctattttatagaacattatcaaatcctttcttccgaagatcagaaaatattactcctaccttagggataaatgtagtacagggagtagggttcattca

SEQ ID NO: 6 - DNA, 432 bp, Bacillus thuringiensis
  misc_feature: Sequence cry1F of Bt from Baja California, Mexico
gcaggaagtgattcatggagagcgccaatgttttcttggacacaccgtagtgcagatcgtacaaatatcattaatccaaatataattacacaaatacctgctgtaaaagcacacaatcttcattcgggttctacggttgttagaggacccgggtttacaggtggtgatctcttacgaagaacgaatactggtacatttgcagatataagagtaaatattactgggccattatctcaaagatatcgtgtaagaattcgctatgcttctacgacagatttacaatttttcacgagaatcaatggaacttctgtaaatcaaggtaatttccaaagaactatgaatagagggggtaatttagagtctggaaactttaggactgcaggatttagtacgccttttagtttttcaaatgcgcaaagtacattcacattg

SEQ ID NO: 7 - DNA, 460 bp, Bacillus thuringiensis
  misc_feature: Sequence cry2Aa of Bt from Baja California, Mexico
caagcgaatataagggagtttaatcaacaagtagataattttttaaaccctactcaaaaccctgttcctttatcaataacttcttcggttaatacaatgcagcaattatttctaaatagattaccccagttccagatacaaggataccagttgttattattacctttatttgcacaggcagccaatatgcatctttcttttattagagatgttattcttaatgcagatgaatggggtatttcagcagcaacattacgtacgtatcgagattacctgagaaattatacaagagattattctaattattgtataaatacgtatcaaactgcgtttagagggttaaacacccgtttacacgatatgttagaatttagaacatatatgtttttaaatgtatttgaatatgtatccatttggtcattgtttaaatatcagagtcttatggtatcttctggcgcta

SEQ ID NO: 8 - DNA, 771 bp, Bacillus thuringiensis
  misc_feature: Sequence cry2Ab of Bt from Baja California, Mexico
cacctggtggagcacgagcttatatggtatctgtgcataacagaaaaaataatatccatgccgttcatgaaaatggttctatgattcatttagcgccaaatgactatacaggatttactatttcgccgatacatgcaactcaagtgaataatcaaacacgaacatttatttctgaaaaatttggaaatcaaggtgattccttaaggtttgaacaaaataacacgacagctcgttatacgcttagagggaatggaaatagttacaatctttatttaagagtttcttcaataggaaattccactattcgagttactataaacggtagggtatatactgctacaaatgttaatactactacaaataacgatggagttaatgataacggagctcgtttttcagatattaatatcggtaatgtagtagcaagtagtaattctgatgtaccattagatataaatgtaacattaaactccggtactcaatttgatcttatgaatattatgcttgtaccaactaatatttcaccactttattaaagtttgaggttcttatgtaaatataagtttatagtttttaatctatctactaaaattaagtatatataatgtatggatgttagaggttgtcttaaagtagttgaatgattactctggggcaacctctttatttttattatcagctggttatattacaaaagaattagaatacttcccagaaaccgataaggtatggattgagattggagaaacggaagggacattcatcgtagac

SEQ ID NO: 9 - DNA, 705 bp, Bacillus thuringiensis
  misc_feature: Sequence cry3 of Bt from Baja California, Mexico
ttaaccgttttcgcagagaaatgacattaactgtattagatctaattgtattattcccattttatgatgttcggttatactcaaaaggagttaaaacagaactaacaagagacatttttacagatccaatttttacactcaatgctcttcaagagtatggaccaactttttcgagtatagaaaactctattcgaaaacctcatttatttgattatttgcgtgggattgaatttcatacgcgtcttcgacctggttactctgggaaagattctttcaattattggtctggtaattatgtagaaactagacctagtataggatctaatgatacaatcacttccccattttatggagataaatctattgaacctatacaaaagctaagctttgatggacaaaaagtttatcgaactatagctaatacagacatagcggcttttccggatggcaagatatattttggtgttacgaaagttgattttagtcaatatgatgatcaaaaaaatgaaactagtacacaaacatatgattcaaaaagatacaatggctatttaggtgcacaggattctatcgaccaattaccaccagaaacaacagatgaaccacttgaaaaagcatatagtcatcagcttaattacgcagaatgtttcttaatgcaggaccgtcgtggaacaattccattttttacttggacacatagaagtg

SEQ ID NO: 10 - DNA, 459 bp, Bacillus thuringiensis
  misc_feature: Sequence cry4 of Bt from Baja California, Mexico
tcaaagatcatttcaaaattacatgtcaacactcaaattttcaacaatcgtattttataagaattcgttttgcttcaaatggaagcgcaaatactcgagctgttataaatcttagtatcccaggggtagcagaactgggtatggcactcaaccccactttttctggtacagattatacgaatttaaaatataaagattttcagtacttagaattttctaacgaggtgaaatttgctccaaatcaaaacatatctcttgtgtttaatcgttcggatgtatatacaaacacaacagtacttattgataaaattgaatttctgccaattactcgttctataagagaggatagagagaaacaaaaattagaaacagtacaacaaataattaatacattttatgcaaatcctataaaaaacactttacaatcagaacttacagattatgacatagatcaagccg

SEQ ID NO: 11 - protein, 132 aa, Bacillus thuringiensis
  MISC_FEATURE: Protein Cry1A of Bt from Baja California, Mexico
FARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPD

SEQ ID NO: 12 - protein, 122 aa, Bacillus thuringiensis
  MISC_FEATURE: Protein Cry1B of Bt from Baja California, Mexico
SSRWSNTRHMTYWRGHTIQSRPIGGGLNTSTHGATNTSINPVTLRFASRDVYRTESYAGVLLWGIYLEPIHGVPTVRFNFTNPQNISDRGTANYSQPYESPGLQLKDSETELPPETTERPNY

SEQ ID NO: 13 - protein, 43 aa, Bacillus thuringiensis
  MISC_FEATURE: Protein Cry1C of Bt from Baja California, Mexico
QRSGTPFLTTGVVFSWTHRSATLTNTIDPERINQIPLVKGFRV

SEQ ID NO: 14 - protein, 213 aa, Bacillus thuringiensis
  MISC_FEATURE: Protein Cry1D of Bt from Baja California, Mexico
REGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSF

SEQ ID NO: 15 - protein, 47 aa, Bacillus thuringiensis
  MISC_FEATURE: Protein Cry1E of Bt from Baja California, Mexico
EPRRTIAPSTFPGLNLFYRTLSNPFFRRSENITPTLGINVVQGVGFI

SEQ ID NO: 16 - protein, 144 aa, Bacillus thuringiensis
  MISC_FEATURE: Protein Cry1F of Bt from Baja California, Mexico
AGSDSWRAPMFSWTHRSADRTNIINPNIITQIPAVKAHNLHSGSTVVRGPGFTGGDLLRRTNTGTFADIRVNITGPLSQRYRVRIRYASTTDLQFFTRINGTSVNQGNFQRTMNRGGNLESGNFRTAGFSTPFSFSNAQSTFTL

SEQ ID NO: 17 - protein, 153 aa, Bacillus thuringiensis
  MISC_FEATURE: Protein Cry2Aa of Bt from Baja California, Mexico
QANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGA

SEQ ID NO: 18 - protein, 175 aa, Bacillus thuringiensis
  MISC_FEATURE: Protein Cry2Ab of Bt from Baja California, Mexico
PGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISP

SEQ ID NO: 19 - protein, 234 aa, Bacillus thuringiensis
  MISC_FEATURE: Protein Cry3 of Bt from Baja California, Mexico
NRFRREMTLTVLDLIVLFPFYDVRLYSKGVKTELTRDIFTDPIFTLNALQEYGPTFSSIENSIRKPHLFDYLRGIEFHTRLRPGYSGKDSFNYWSGNYVETRPSIGSNDTITSPFYGDKSIEPIQKLSFDGQKVYRTIANTDIAAFPDGKIYFGVTKVDFSQYDDQKNETSTQTYDSKRYNGYLGAQDSIDQLPPETTDEPLEKAYSHQLNYAECFLMQDRRGTIPFFTWTHRS

SEQ ID NO: 20 - protein, 152 aa, Bacillus thuringiensis
  MISC_FEATURE: Protein Cry4 of Bt from Baja California, Mexico
KDHFKITCQHSNFQQSYFIRIRFASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQA

SEQ ID NO: 21 - DNA, 18 bp, Artificial Sequence
  Primer forward. Amplifies gene cry1A
attcgctaggaaccaagc

SEQ ID NO: 22 - DNA, 18 bp, Artificial Sequence
  Primer reverse. Amplifies gene cry1A
aatccggtccccatacac

SEQ ID NO: 23 - DNA, 19 bp, Artificial Sequence
  Primer forward. Amplifies gene cry1B
cttcatcacgatggagtaa

SEQ ID NO: 24 - DNA, 20 bp, Artificial Sequence
  Primer reverse. Amplifies gene cry1B
cataatttggtcgttctgtt

SEQ ID NO: 25 - DNA, 19 bp, Artificial Sequence
  misc_feature: Primer forward. Amplifies gene cry1C
caaagatctggaacacctt

SEQ ID NO: 26 - DNA, 20 bp, Artificial Sequence
  Primer reverse. Amplifies gene cry1C
caaactctaaatcctttcac

SEQ ID NO: 27 - DNA, 20 bp, Artificial Sequence
  Primer forward. Amplifies gene cry1D
aagggaaggaaatacagagc

SEQ ID NO: 28 - DNA, 19 bp, Artificial Sequence
  Primer reverse. Amplifies gene cry1D
cgaacgaacgagatgttag

SEQ ID NO: 29 - DNA, 19 bp, Artificial Sequence
  Primer forward. Amplifies gene cry1E
gaaccaagacgaactattg

SEQ ID NO: 30 - DNA, 18 bp, Artificial Sequence
  Primer reverse. Amplifies gene cry1E
tgaatgaaccctactccc

SEQ ID NO: 31 - DNA, 18 bp, Artificial Sequence
  Primer forward. Amplifies gene cry1F
gcaggaagtgattcatgg

SEQ ID NO: 32 - DNA, 20 bp, Artificial Sequence
  Primer reverse. Amplifies gene cry1F
caatgtgaatgtactttgcg

SEQ ID NO: 33 - DNA, 19 bp, Artificial Sequence
  Primer forward. Amplifies gene cry2Aa
caagcgaatataagggagt

SEQ ID NO: 34 - DNA, 18 bp, Artificial Sequence
  Primer reverse. Amplifies gene cry2Aa
tagcgccagaagatacca

SEQ ID NO: 35 - DNA, 18 bp, Artificial Sequence
  Primer forward. Amplifies gene cry2Ab
cacctggtggagcacgag

SEQ ID NO: 36 - DNA, 18 bp, Artificial Sequence
  Primer reverse. Amplifies gene cry2Ab
gtctacgatgaatgtccc

SEQ ID NO: 37 - DNA, 19 bp, Artificial Sequence
  Primer forward. Amplifies gene cry3
ttaaccgttttcgcagaga

SEQ ID NO: 38 - DNA, 22 bp, Artificial Sequence
  Primer reverse. Amplifies gene cry3
tccgcacttctatgtgtccaag

SEQ ID NO: 39 - DNA, 25 bp, Artificial Sequence
  Primer forward. Amplifies gene cry4
tcaaagatcatttcaaaattacatg

SEQ ID NO: 40 - DNA, 25 bp, Artificial Sequence
  Primer reverse. Amplifies gene cry4
cggcttgatctatgtcataatctgt